(19) United States
(12) Patent Application Publication
(10) Pub. No.: US 2024/0324936 A1
(43) Pub. Date: Oct. 3, 2024

(54) ELECTROCARDIOGRAPH (ECG) SIGNAL ENHANCEMENT METHOD BASED ON NOVEL GENERATIVE ADVERSARIAL NETWORK (GAN)

(71) Applicants: Qilu University of Technology (Shandong Academy of Sciences), Jinan (CN); SHANDONG COMPUTER SCIENCE CENTER (NATIONAL SUPERCOMPUTING CENTER IN JINAN), Jinan (CN)

(72) Inventors: Yinglong WANG, Jinan (CN); Tiantian DU, Jinan (CN); Minglei SHU, Jinan (CN); Zhaoyang LIU, Jinan (CN); Shuwang ZHOU, Jinan (CN); Pengyao XU, Jinan (CN)

(73) Assignees: Qilu University of Technology (Shandong Academy of Sciences), Jinan (CN); SHANDONG COMPUTER SCIENCE CENTER (NATIONAL SUPERCOMPUTING CENTER IN JINAN), Jinan (CN)

(21) Appl. No.: 18/525,893

(22) Filed: Dec. 1, 2023

(30) Foreign Application Priority Data
Apr. 3, 2023 (CN) 202310340465.0

(51) Int. Cl. A61B 5/352; A61B 5/00; G06N 3/045

(52) U.S. Cl. CPC A61B 5/352; A61B 5/7203; G06N 3/045

(57) ABSTRACT

An electrocardiograph (ECG) signal enhancement method based on a novel generative adversarial network (GAN) effectively enhances a capability of a generator model for understanding and expressing input data by using a multi-branch structure of bi-directional long short-term memory (BiLSTM) neural networks with different quantities of hidden neurons, and stitching outputs of last time steps of forward propagation of the different BiLSTM networks. A new ECG signal enhancement module EEA-Net is proposed, which uses an adaptive convolutional layer to dynamically adjust a size of a convolution kernel, making the model more flexible in processing input sequences of different lengths. In addition, the model uses an adaptive average pooling layer to perform weighted average pooling on the input data to better capture important information of the input data.

### CROSS REFERENCE TO THE RELATED APPLICATIONS

This application is based upon and claims priority to Chinese Patent Application No. 202310340465.0, filed on Apr. 3, 2023, the entire contents of which are incorporated herein by reference.

### TECHNICAL FIELD

The present disclosure relates to the technical field of electrocardiograph (ECG) signal processing, and specifically, to an ECG signal enhancement method based on a novel generative adversarial network (GAN).

### BACKGROUND

At present, due to a serious imbalance between normal and abnormal signals in an existing ECG database, an existing ECG signal classification method has an unsatisfactory classification effect for an imbalanced ECG dataset. Therefore, an effective ECG signal enhancement technique can help improve a classification effect of imbalanced ECG data.

Traditionally, an ECG signal is augmented mainly by means of a mathematical model, translation, noising, and other methods. An existing deep learning method generates the ECG signal mainly by using an oversampling technique, a codec structure, and the like. However, these methods do not achieve an expected enhancement effect for the ECG signal and have no objective standard for determining quality of the generated ECG signal. As one of the widely used data augmentation methods now, a GAN has also been applied in the field of ECG signal processing. However, because some categories in a current imbalanced ECG signal dataset have a relatively small amount of data, there are certain difficulties in synthesizing high-quality data by using a GAN model.

### SUMMARY

In order to overcome the shortcomings in the above technologies, the present disclosure provides an ECG signal enhancement method based on a novel GAN.

The technical solutions used in the present disclosure to resolve the technical problems thereof are as follows:

An ECG signal enhancement method based on a novel GAN includes the following steps:
a) resampling a collected ECG signal;
b) normalizing resampled ECG signal data;
c) extracting a location of an R-wave of a normalized ECG signal by using a Pan_Tompkin detection algorithm, dividing the normalized ECG signal into heartbeats based on m sampling points before the R-wave and n sampling points after the R-wave to obtain a heartbeat dataset U={U₁, U₂, . . . , Uₜ, . . . , U_{Z}}, where Uₜ represents a t^{th} heartbeat record, tϵ{1, 2, . . . , Z}, Z represents a quantity of the heartbeats, a length of each heartbeat record is represented by N, and each heartbeat record has 1*N dimensions;
d) dividing the heartbeat dataset U into a training set Xₜᵣₐᵢₙ, a validation set Xᵥₐₗ, and a test set Xₜₑₛₜ in a ratio of a:b:c;
e) establishing a GAN model including a generator network and a discriminator network, and inputting random noise Noise_{fixed} into the generator network of the GAN model in batches to output a synthesized signal set X_{g}, where the random noise Noise_{fixed} has batch*1*N dimensions;
f) inputting the training set Xₜᵣₐᵢₙ into the discriminator network of the GAN model to obtain an output label Xₜᵣₐᵢₙ^{d}, and inputting the synthesized signal set X_{g} into the discriminator network of the GAN model to obtain an output label X_{g}^{d};
g) training the generator network and the discriminator network by means of a game; and
h) inputting the random noise N_{g} with the batch*1*N dimensions into a trained generator network to obtain an enhanced ECG signal U_{g}.

Preferably, a sampling frequency during the resampling in the step a) is 125 Hz.

Preferably, in the step d), a=7, b=1.5, and c=1.5.

Further, the step e) includes the following substeps:
e-1) constituting the generator network by a first convolutional block, a second convolutional block, a third convolutional block, a multi-branch bi-directional long short-term memory (BiLSTM) neural network module, an EEA-Net module, and a fully connected layer in sequence;
e-2) constituting the first convolutional block by a convolutional layer, a BatchNorm1d layer, and a Swish activation function layer in sequence, inputting the random noise Noise_{fixed} into the convolutional layer of the first convolutional block to obtain a feature X₁₋₁, inputting the feature X₁₋₁ into the BatchNorm1d layer of the first convolutional block to obtain a feature X₁₋₂, and inputting the feature X₁₋₂ into the Swish activation function layer of the first convolutional block to obtain a feature X₁;
e-3) constituting the second convolutional block by a convolutional layer, a BatchNorm1d layer, and a Swish activation function layer in sequence, inputting the feature X₁ into the convolutional layer of the second convolutional block to obtain a feature X₂₋₁, inputting the feature X₂₋₁ into the BatchNorm1d layer of the second convolutional block to obtain a feature X₂₋₂, and inputting the feature X₂₋₂ into the Swish activation function layer of the second convolutional block to obtain a feature X₂;
e-4) constituting the third convolutional block by a convolutional layer, a BatchNorm1d layer, a Swish activation function layer, and a Max-pooling layer in sequence, inputting the feature X₂ into the convolutional layer of the third convolutional block to obtain a feature X₃₋₁, inputting the feature X₃₋₁ into the BatchNorm1d layer of the third convolutional block to obtain a feature X₃₋₂, stitching the feature X₃₋₂ and the feature X₁₋₂, inputting a stitched feature into the Swish activation function layer of the third convolutional block to obtain a feature X₃₋₃, and inputting the feature X₃₋₃ into the Max-pooling layer of the third convolutional block to obtain a feature X₃;
e-5) constituting the multi-branch BiLSTM neural network module of the generator network by a first BiLSTM network, a second BiLSTM network, and a third BiLSTM network; inputting the feature X₃ into the first BiLSTM network, and calculating an output X^{bilstm1} of the first BiLSTM network according to a formula X^{bilstm1}=concat(h_{L,1},h_{R,1}), where concat(·) represents a stitching operation, h_{L,1} represents a hidden state of a last time step of forward propagation of the first BiLSTM network, and h_{R,1} represents a hidden state of a last time step of backward propagation of the first BiLSTM network; inputting the feature X₃ into the second BiLSTM network, and calculating an output X^{bilstm2} of the second BiLSTM network according to a formula X^{bilstm2}=concat(concat(h_{L,1},h_{L,2}),h_{R,2}), where h_{L,2} represents a hidden state of a last time step of forward propagation of the second BiLSTM network, and h_{R,2} represents a hidden state of a last time step of backward propagation of the second BiLSTM network; inputting the feature X₃ into the third BiLSTM network, and calculating an output X^{bilstm3} of the third BiLSTM network according to a formula X^{bilstm3}=concat(concat(h_{L,2},h_{L,3}),h_{R,3}), where h_{L,3} represents a hidden state of a last time step of forward propagation of the third BiLSTM network, and h_{R,3} represents a hidden state of a last time step of backward propagation of the third BiLSTM network; and calculating a final output feature X₄ of the multi-branch BiLSTM neural network module according to a formula X₄=concat(X^{bilstm1},X^{bilstm2},X^{bilstm3});
e-6) constituting the EEA-Net module of the generator network by a first convolutional block, a second convolutional block, an adaptive average pooling layer, a third convolutional block, and a fully connected module, where the first convolutional block is constituted by a convolutional layer and a LeakyReLU activation function layer in sequence, the second convolutional block is constituted by an adaptive convolutional layer and a LeakyReLU activation function layer in sequence, the third convolutional block is constituted by a convolutional layer and a LeakyReLU activation function layer in sequence, and the fully connected module is constituted by a fully connected layer and a Sigmoid activation function layer in sequence;
e-7) inputting the feature X₄ into the first convolutional block of the EEA-Net module of the generator network to obtain a feature X₄₋₁, inputting the feature X₄ into the second convolutional block of the EEA-Net module of the generator network to obtain a feature X₄₋₂, stitching the feature X₄₋₁ and the feature X₄₋₂ to obtain a stitching result C₁, inputting the stitching result C₁ into the adaptive average pooling layer, the third convolutional block, and the fully connected module of the EEA-Net module in sequence to obtain feature information X_{E}, and performing point multiplication on the feature information X_{E} and the feature X₄ to obtain a final output feature X₅ of the EEA-Net module; and
e-8) inputting the output feature X₅ into the fully connected layer of the generator network to obtain the synthesized signal set X_{g}.

Preferably, the convolutional layer of the first convolutional block in the step e-2) has a 1×3 convolution kernel and a stride of 1, and a padding of 1; the convolutional layer of the second convolutional block in the step e-3) has a 1×5 convolution kernel and a stride of 2, and a padding of 2; the convolutional layer of the third convolutional block in the step e-4) has a 1×3 convolution kernel and a stride of 1, and a padding of 1; in the step e-5), the first BiLSTM network has 64 neurons, the second BiLSTM network has 128 neurons, and the third BiLSTM network has 256 neurons; the convolutional layer of the first convolutional block in the step e-6) has a 1×3 convolution kernel and a stride of 1, and a padding of 1; the adaptive convolutional layer of the second convolutional block in the step e-6) has a stride of 1, and a padding of 1; and the convolutional layer of the third convolutional block in the step e-6) has a 1×5 convolution kernel and a stride of 1, and a padding of 1.

Further, the step b) includes the following substeps:
f-1) constituting the discriminator network of the GAN model by a BiLSTM network, a first fully connected layer, a first Swish activation function layer, a second fully connected layer, a second Swish activation function layer, a Dropout layer, a third fully connected layer, and a Sigmoid layer in sequence;
f-2) inputting the training set Xₜᵣₐᵢₙ into the BiLSTM network of the discriminator network in batches to obtain a feature X₆, and inputting the feature X₆ into the first fully connected layer, the first Swish activation function layer, the second fully connected layer, the second Swish activation function layer, the Dropout layer, the third fully connected layer, and the Sigmoid layer of the discriminator network in sequence to obtain the output label Xₜᵣₐᵢₙ^{d}; and
f-3) inputting the synthesized signal set X_{g} into the BiLSTM network of the discriminator network in batches to obtain a feature X₇, and inputting the feature X₇ into the first fully connected layer, the first Swish activation function layer, the second fully connected layer, the second Swish activation function layer, the Dropout layer, the third fully connected layer, and the Sigmoid layer of the discriminator network in sequence to obtain the output label X_{g}^{d}.

Preferably, in the step f-1), the BiLSTM network has 128 hidden neurons, and the Dropout layer has a random dropout rate of 0.2.

Further, the step g) includes the following substeps:
g-1) constructing a real label Lᵣₑₐₗ containing only is, and a false label L_{fake} containing only 0s;
g-2) calculating a loss function loss_{D} of the discriminator network according to a following formula:

$${loss}_{D} = - ( {L}_{real} * log ( {X}_{train}^{d} ) + ( 1 - {L}_{real} ) * log ( 1 - {X}_{train}^{d} ) ) - ( {L}_{fake} * log ( {X}_{g}^{d} ) + ( 1 - {L}_{fake} ) * log ( 1 - {X}_{g}^{d} ) ) ;$$

g-3) calculating a loss function loss_{G} of the generator network according to a following formula:

$${loss}_{G} = β [ - ( {L}_{real} * log ( {X}_{g}^{d} ) + ( 1 - {L}_{real} ) * log ( 1 - {X}_{g}^{d} ) ) ] + γ [ \frac{1}{K} {\sum }_{i = 1}^{K} {( {X}_{train}^{i} - {X}_{g}^{i} )}^{2} ]$$

where both β and γ are equilibrium factors, Xₜᵣₐᵢₙⁱ represents an i^{th}, heartbeat record in the training set Xₜᵣₐᵢₙ, X_{g}ⁱ represents an i^{th} record in the synthesized signal set X_{g}, iϵ{1, 2, . . . , K}, and

$$K = \frac{Za}{a + b + c} ;$$

and
g-4) using an Adam optimizer to train the generator network based on the loss function loss_{G} of the generator network, and train the discriminator network based on the loss function loss_{D} of the discriminator network, to obtain an optimized GAN model.

Preferably, β=0.7, and γ=0.2.

Preferably, during the training in the step g-4), a learning rate, a batch size, and an iteration quantity are respectively set to 0.0002, 128, and 2500 for both the generator network and the discriminator network, and the learning rates of the generator network and the discriminator network are updated to 0.1 times the original ones every 500 iterations.

The present disclosure has following beneficial effects: A capability of a generator model for understanding and expressing input data is effectively enhanced by using a multi-branch structure of BiLSTM neural networks with different quantities of hidden neurons, and stitching outputs of last time steps of forward propagation of the different BiLSTM networks. The present disclosure provides a new ECG signal enhancement module EEA-Net in a generator, which uses an adaptive convolutional layer to dynamically adjust a size of a convolution kernel, making the model more flexible in processing input sequences of different lengths. In addition, the model uses an adaptive average pooling layer to perform weighted average pooling on the input data to better capture important information of the input data. After processing by a convolutional layer, a fully connected layer, and the like, the model performs point multiplication on the obtained information and the original data to focus on important feature information of an ECG signal. Game training is performed on the generator and a discriminator that are constituted by combining a multi-branch BiLSTM neural network and an EEA-Net. In this way, the generator can better capture the important feature information of the ECG signal, which is conducive to generating a high-quality ECG signal.

### BRIEF DESCRIPTION OF THE DRAWINGS

FIG. 1 is a structural diagram of a GAN model according to the present disclosure;

FIG. 2 is a structural diagram of a multi-branch BiLSTM neural network module according to the present disclosure; and

FIG. 3 is a structural diagram of an EEA-Net module according to the present disclosure.

FIG. 4 shows comparison between some real signals and synthesized signals of the category S.

FIG. 5 shows comparison between some real signals and synthesized signals of the category F.

### DETAILED DESCRIPTION OF THE EMBODIMENTS

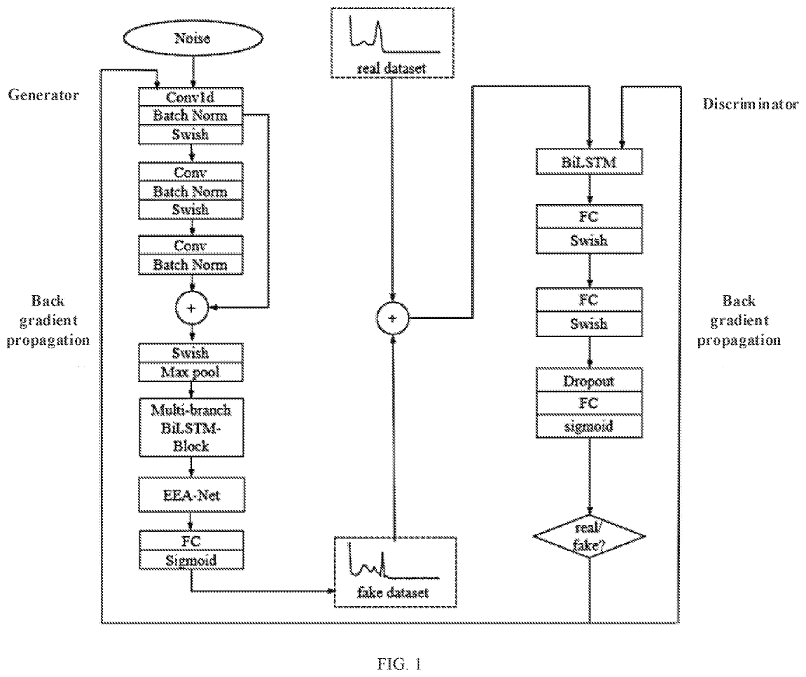

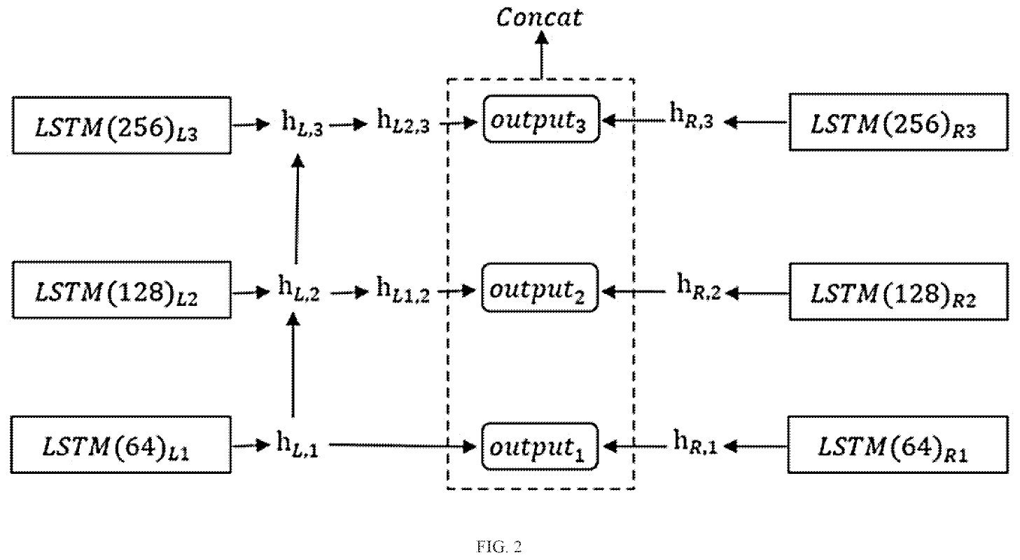

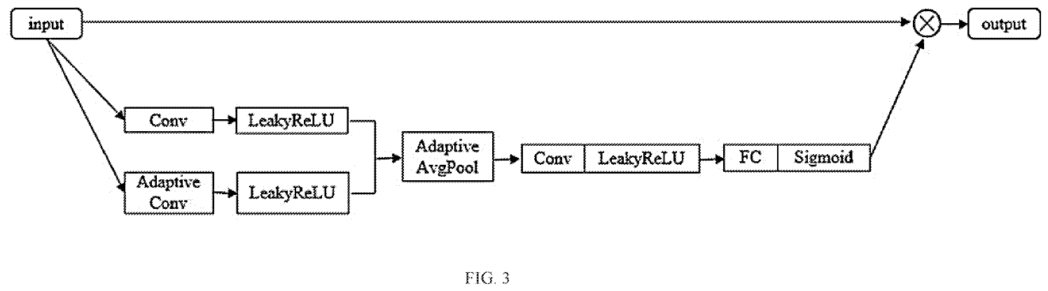

The present disclosure is further described below with reference to FIG. 1, FIG. 2, and FIG. 3.

An ECG signal enhancement method based on a novel GAN includes the following steps:
a): A collected ECG signal is resampled. In a specific embodiment of the present disclosure, preferably, a sampling frequency during the resampling in the step a) is 125 Hz.
b): Resampled ECG signal data is normalized.
c): A location of an R-wave of a normalized ECG signal is extracted by using a Pan_Tompkin detection algorithm, the normalized ECG signal is divided into heartbeats based on m sampling points before the R-wave and n sampling points after the R-wave to obtain heartbeat dataset U={U₁, U₂, . . . , Uₜ, . . . , U_{Z}}, where Uₜ represents a t^{th} heartbeat record, tϵ{1, 2, . . . , Z}, Z represents a quantity of the heartbeats, a length of each heartbeat record is represented by N, and each heartbeat record has 1*N dimensions.
d): The heartbeat dataset U is divided into training set Xₜᵣₐᵢₙ, validation set Xᵥₐₗ, and test set Xₜₑₛₜ in a ratio of a:b:c. In a specific embodiment of the present disclosure, preferably, a=7, b=1.5, and c=1.5.
e): A GAN model including a generator network and a discriminator network is established, and random noise Noise_{fixed} is input into the generator network of the GAN model in batches to output synthesized signal set X_{g}. The random noise Noise_{fixed} has batch*1*N dimensions.
f): The training set Xₜᵣₐᵢₙ is input into the discriminator network of the GAN model to obtain output label Xₜᵣₐᵢₙ^{d}, and the synthesized signal set X_{g} is input into the discriminator network of the GAN model to obtain output label X_{g}^{d}.
g): The generator network and the discriminator network are trained by means of a game.
h): The random noise N_{g} with the batch*1*N dimensions is input into a trained generator network to obtain enhanced ECG signal U_{g}.

The present disclosure provides an ECG signal enhancement method based on a novel GAN. A generator of a GAN model uses a multi-branch BiLSTM neural network and an ECG signal enhancement network (EEA-Net) to capture an underlying feature of an ECG signal and enhance the ECG signal, thereby generating a high-quality ECG signal. Then, a discriminator is used to determine a real ECG signal and a synthesized ECG signal, and probabilities of being the real ECG signal and the synthesized ECG signal. Through game optimization of the generator and the discriminator, a GAN can be configured to generate high-quality ECG signal data, providing strong support for classifying imbalanced ECG data.

### Embodiment 1

The step e) includes the following substeps:
e-1): The generator network is constituted by a first convolutional block, a second convolutional block, a third convolutional block, a multi-branch BiLSTM neural network module, an EEA-Net module, and a fully connected layer in sequence.
e-2): The first convolutional block is constituted by a convolutional layer, a BatchNorm1d layer, and a Swish activation function layer in sequence, the random noise Noise_{fixed} is input into the convolutional layer of the first convolutional block to obtain feature X₁₋₁, the feature X₁₋₁ is input into the BatchNorm1d layer of the first convolutional block to obtain feature X₁₋₂, and the feature X₁₋₂ is input into the Swish activation function layer of the first convolutional block to obtain feature X₁.
e-3): The second convolutional block is constituted by a convolutional layer, a BatchNorm1d layer, and a Swish activation function layer in sequence, the feature X₁ is input into the convolutional layer of the second convolutional block to obtain feature X₂₋₁, the feature X₂₋₁ is input into the BatchNorm1d layer of the second convolutional block to obtain feature X₂₋₂, and the feature X₂₋₂ is input into the Swish activation function layer of the second convolutional block to obtain feature X₂.
e-4): The third convolutional block is constituted by a convolutional layer, a BatchNorm1d layer, a Swish activation function layer, and a Max-pooling layer in sequence, the feature X₂ is input into the convolutional layer of the third convolutional block to obtain feature X₃₋₁, the feature X₃₋₁ is input into the BatchNorm1d layer of the third convolutional block to obtain feature X₃₋₂, the feature X₃₋₂ and the feature X₁₋₂ are stitched, a stitched feature is input into the Swish activation function layer of the third convolutional block to obtain feature X₃₋₃, and the feature X₃₋₃ is input into the Max-pooling layer of the third convolutional block to obtain feature X₃.
e-5): The multi-branch BiLSTM neural network module of the generator network is constituted by a first BiLSTM network, a second BiLSTM network, and a third BiLSTM network; the feature X₃ is input into the first BiLSTM network, and output X^{bilstm1} of the first BiLSTM network is calculated according to formula X^{bilstm1}=concat(h_{L,1},h_{R,1}), where concat(·) represents a stitching operation, h_{L,1} represents a hidden state of a last time step of forward propagation of the first BiLSTM network, and h_{R,1} represents a hidden state of a last time step of backward propagation of the first BiLSTM network; the feature X₃ is input into the second BiLSTM network, and output X^{bilstm2} of the second BiLSTM network is calculated according to formula X^{bilstm2}=concat(concat(h_{L,1},h_{L,2}),h_{R,2}), where h_{L,2} represents a hidden state of a last time step of forward propagation of the second BiLSTM network, and h_{R,2} represents a hidden state of a last time step of backward propagation of the second BiLSTM network; and the feature X₃ is input into the third BiLSTM network, and output X^{bilstm3} of the third BiLSTM network is calculated according to formula X^{bilstm3}=concat(concat(h_{L,2},h_{L,3}) h_{R,3}), where h_{L,3} represents a hidden state of a last time step of forward propagation of the third BiLSTM network, and h_{R,3} represents a hidden state of a last time step of backward propagation of the third BiLSTM network. A final output of the multi-branch BiLSTM neural network module is a stitching result of the outputs of the three BiLSTM networks on a third dimension. Specifically, final output feature X₄ of the multi-branch BiLSTM neural network module is calculated according to formula

$${X}_{4} = concat ( {X}^{bilstm 1} , {X}^{bilstm 2} , {X}^{bilstm 3} ) .$$

e-6): The EEA-Net module of the generator network is constituted by a first convolutional block, a second convolutional block, an adaptive average pooling layer, a third convolutional block, and a fully connected module, where the first convolutional block is constituted by a convolutional layer and a LeakyReLU activation function layer in sequence, the second convolutional block is constituted by an adaptive convolutional layer and a LeakyReLU activation function layer in sequence, the third convolutional block is constituted by a convolutional layer and a LeakyReLU activation function layer in sequence, and the fully connected module is constituted by a fully connected layer and a Sigmoid activation function layer in sequence.
e-7): The feature X₄ is input into the first convolutional block of the EEA-Net module of the generator network to obtain feature X₄₋₁, the feature X₄ is input into the second convolutional block of the EEA-Net module of the generator network to obtain feature X₄₋₂, the feature X₄₋₁ and the feature X₄₋₂ are stitched to obtain stitching result C₁, the stitching result C₁ is input into the adaptive average pooling layer, the third convolutional block, and the fully connected module of the EEA-Net module in sequence to obtain feature information X_{E}, and point multiplication is performed on the feature information X_{E} and the feature X₄ to obtain final output feature X₅ of the EEA-Net module.

### Embodiment 2

e-8): The output feature X₅ is input into the fully connected layer of the generator network to obtain the synthesized signal set X_{g}.

Further, preferably, the convolutional layer of the first convolutional block in the step e-2) has a 1×3 convolution kernel and a stride of 1, and a padding of 1. The convolutional layer of the second convolutional block in the step e-3) has a 1×5 convolution kernel and a stride of 2, and a padding of 2. The convolutional layer of the third convolutional block in the step e-4) has a 1×3 convolution kernel and a stride of 1, and a padding of 1. In the step e-5), the first BiLSTM network has 64 neurons, the second BiLSTM network has 128 neurons, and the third BiLSTM network has 256 neurons. The convolutional layer of the first convolutional block in the step e-6) has a 1×3 convolution kernel and a stride of 1, and a padding of 1. The adaptive convolutional layer of the second convolutional block in the step e-6) has a stride of 1 and a convolution kernel whose size can be adaptively adjusted based on characteristics of the input data, and a padding of 1. A size of a pooling kernel in the adaptive average pooling layer can also be adaptively adjusted based on the characteristics of the input data. The convolutional layer of the third convolutional block in the step e-6) has a 1×5 convolution kernel and a stride of 1, and a padding of 1.

### Embodiment 3

The step f) includes the following substeps:
f-1): The discriminator network of the GAN model is constituted by a BiLSTM network, a first fully connected layer, a first Swish activation function layer, a second fully connected layer, a second Swish activation function layer, a Dropout layer, a third fully connected layer, and a Sigmoid layer in sequence. In an embodiment of the present disclosure, preferably, in the step f-1), the BiLSTM network has 128 hidden neurons, and the Dropout layer has a random dropout rate of 0.2.
f-2): The training set Xₜᵣₐᵢₙ is input into the BiLSTM network of the discriminator network in batches to obtain feature X₆, and the feature X₆ is input into the first fully connected layer, the first Swish activation function layer, the second fully connected layer, the second Swish activation function layer, the Dropout layer, the third fully connected layer, and the Sigmoid layer of the discriminator network in sequence to obtain the output label Xₜᵣₐᵢₙ^{d}. The output label Xₜᵣₐᵢₙ^{d} is a set containing 0 and 1. 0 and 1 are labels output when whether an input signal is true or false is determined. If the input signal is true, 1 is output. If the input signal is false, 0 is output.
f-3): The synthesized signal set X_{g} is input into the BiLSTM network of the discriminator network in batches to obtain feature X₇, and the feature X₇ is input into the first fully connected layer, the first Swish activation function layer, the second fully connected layer, the second Swish activation function layer, the Dropout layer, the third fully connected layer, and the Sigmoid layer of the discriminator network in sequence to obtain the output label X_{g}^{d}. The output label X_{g}^{d} is a set containing 0 and 1. 0 and 1 are labels output when whether an input signal is true or false is determined. If the input signal is true, 1 is output. If the input signal is false, 0 is output.

### Embodiment 4

The step g) includes the following substeps:
g-1): A real label Lᵣₑₐₗ containing only 1s and a false label L_{fake} containing only 0s are constructed.
g-2): A loss of the discriminator is calculated. The loss of the discriminator is a sum of a difference between an output label of input real data and the real label, and a cross-entropy loss between an output label of input false data and the false label during the training.

Specifically, loss function loss_{D} of the discriminator network is calculated according to a following formula:

$${loss}_{D} = - ( {L}_{real} * log ( {X}_{train}^{d} ) + ( 1 - {L}_{real} ) * log ( 1 - {X}_{train}^{d} ) ) - ( {L}_{fake} * log ( {X}_{g}^{d} ) + ( 1 - {L}_{fake} ) * log ( 1 - {X}_{g}^{d} ) )$$

g-3): A loss of the generator is calculated. The loss of the generator is a sum of a cross-entropy loss between a result of determining a synthesized signal by the discriminator and the real label, and an MSE loss between data synthesized by the generator and the real data. Specifically, loss function loss_{G} of the generator network is calculated according to a following formula:

$${loss}_{G} = β [ - ( {L}_{real} * log ( {X}_{g}^{d} ) + ( 1 - {L}_{real} ) * log ( 1 - {X}_{g}^{d} ) ) ] + γ [ \frac{1}{K} {\sum }_{i = 1}^{K} {( {X}_{train}^{i} - {X}_{g}^{i} )}^{2} ] ,$$

where both β and γ are equilibrium factors, Xₜᵣₐᵢₙⁱ represents an i^{th} heartbeat record in the training set Xₜᵣₐᵢₙ, X_{g}ⁱ represents an i^{th} record in the synthesized signal set X_{g}, iϵ{1, 2, . . . , K}, and

$$K = \frac{Za}{a + b + c} .$$

### Preferably, β=0.7, and γ=0.2.

g-4) An Adam optimizer is used to train the generator network based on the loss function loss_{G} of the generator network, and train the discriminator network based on the loss function loss_{D} of the discriminator network, to obtain an optimized GAN model. In a specific embodiment of the present disclosure, during the training in the step g-4), a learning rate, a batch size, and an iteration quantity are respectively set to 0.0002, 128, and 2500 for both the generator network and the discriminator network, and the learning rates of the generator network and the discriminator network are updated to 0.1 times the original ones every 500 iterations.

Implementations of the present disclosure are specifically described below by taking data in a public dataset MIT-BIH as an example.

ECG signal data and corresponding labels are obtained from the MIT-BIH dataset (classified into categories N, V, Q, S, and F) to construct an ECG signal enhancement model of a novel GAN. The model is constituted by a data preprocessing module, a generator network module (including a first convolutional block, a second convolutional block, a third convolutional block, a multi-branch BiLSTM neural network module, an EEA-Net module, and a fully connected layer), and a discriminator network module based on a BiLSTM neural network. An ECG signal is preprocessed. Specifically, resampling is performed, the ECG signal is then extracted before and after an R-peak, and ECG signal data of 187 sample points is finally obtained.

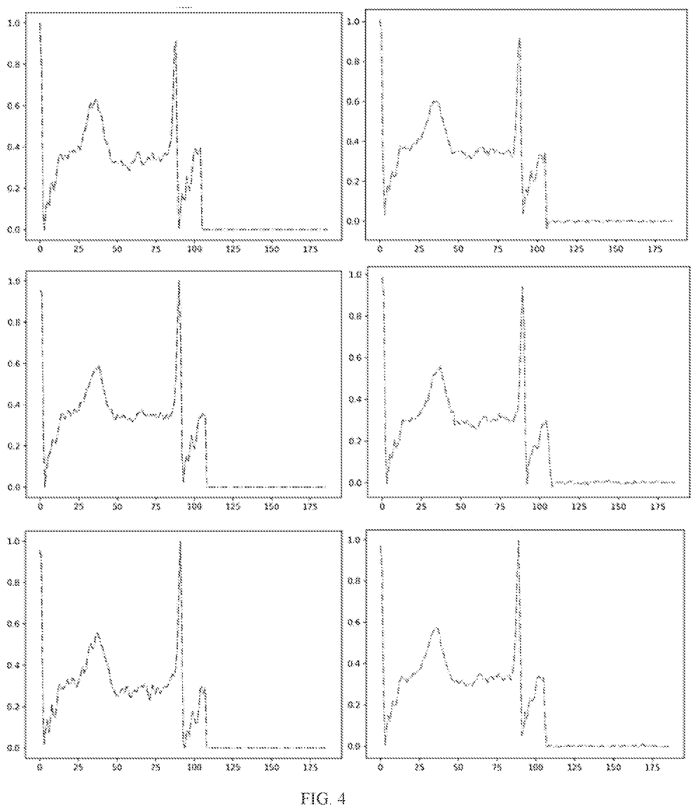

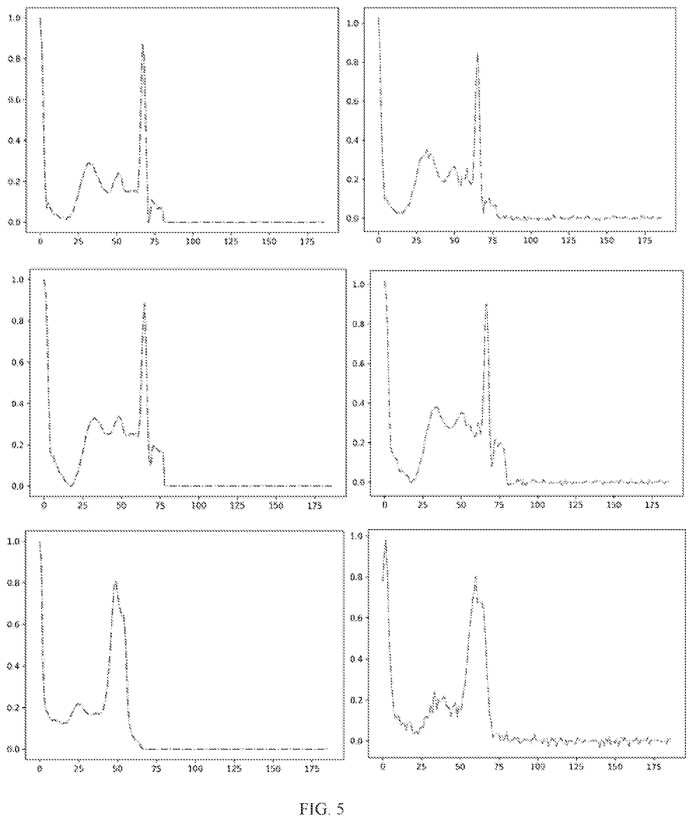

The MIT-BIH dataset is used for training, and an ECG signal synthesized by the model is presented by using a sequence chart. In addition, effectiveness of the model is demonstrated by comparing the model with various models (DCGAN, LSGAN, LSTM-GAN, WGAN, and CRNN-GAN) based on quantitative evaluation indicators. FIG. 4 and FIG. 5 visually compare some original signals and synthesized signals of the categories S and F in the MIT-BIH dataset. As shown in the figures, the original signals and the synthesized signals are highly consistent in morphology, which indicates effectiveness of the model provided in the present disclosure in capturing an ECG signal feature.

Table 1 shows a result of comparing the method and other models based on seven quantitative evaluation indicators. Under all the evaluation indicators, the model provided in the present disclosure exhibits superior performance compared with other models, which proves that the method provided in the present disclosure can achieve better generation performance and generate a higher-quality ECG signal.

As shown in Table 2, Table 3, and Table 4, in addition to using the above evaluation indicators to evaluate quality of the generated ECG signal, a comparative experiment on a classification task is also conducted. Classification by augmenting synthesized ECG signals of the categories S and F with a small amount of data into an original dataset is compared with classification by using only the original dataset. Through this comparison, an impact of the synthesized ECG signal on performance of a classification model can be objectively evaluated. In the present disclosure, three deep learning classification models are used to conduct an experiment, and precision, a recall rate, an F1 score, and accuracy are selected as evaluation indicators. From the tables, it can be concluded that regardless of which classification model is used, on a premise of ensuring a slight improvement in overall classification accuracy or not changing the overall classification accuracy, data augmentation for categories with a little sample data (categories S and F) can effectively improve an ECG signal classification effect for the categories with a little sample data (categories S and F).

TABLE 1
Analysis on quantitative evaluation indicators for
a signal generated based on the MIT-BIH dataset

| | MSE | RMSE | MAPE | MAE | DTW | PRD | ED |
|---|---|---|---|---|---|---|---|
| LMD-GAN | 0.022 | 0.149 | 1.4319 | 0.0655 | 4.873 | 6.628 | 1.7623 |
| DCGAN | 0.029 | 0.170 | 1.019 | 0.104 | 11.245 | 14.565 | 2.183 |
| LSGAN | 0.027 | 0.164 | 1.643 | 0.075 | 5.579 | 7.029 | 1.997 |
| LSTM-GAN | 0.028 | 0.167 | 1.75 | 0.108 | 12.9 | 7.081 | 2.153 |
| WGAN | 0.028 | 0.167 | 1.585 | 0.082 | 6.361 | 7.209 | 2.049 |
| CRNN-GAN | 0.174 | 0.417 | 3.535 | 0.313 | 52.073 | 10.835 | 5.673 |

TABLE 2
Comprehensive classification performance
when a CNN model is used

| | | N | V | Q | S | F | Acc |
|---|---|---|---|---|---|---|---|
| Original | precision | 0.995 | 0.975 | 0.992 | 0.719 | 0.720 | 0.984 |
| | recall | 0.989 | 0.947 | 0.997 | 0.866 | 0.935 | |
| | F1-score | 0.992 | 0.961 | 0.994 | 0.786 | 0.814 | |
| Augmented | precision | 0.997 | 0.966 | 0.992 | 0.862 | 0.936 | 0.987 |
| | recall | 0.990 | 0.972 | 0.991 | 0.975 | 0.965 | |
| | F1-score | 0.993 | 0.969 | 0.992 | 0.915 | 0.950 | |

TABLE 3
Comprehensive classification performance
when an RNN model is used

| | | N | V | Q | S | F | Acc |
|---|---|---|---|---|---|---|---|
| Original | precision | 0.998 | 0.963 | 0.989 | 0.450 | 0.783 | |
| | recall | 0.981 | 0.958 | 0.983 | 0.996 | 0.887 | 0.979 |
| | F1-score | 0.989 | 0.960 | 0.986 | 0.620 | 0.832 | |
| Augmented | precision | 0.997 | 0.969 | 0.991 | 0.858 | 0.927 | |
| | recall | 0.989 | 0.959 | 0.996 | 0.974 | 0.968 | 0.987 |
| | F1-score | 0.993 | 0.964 | 0.993 | 0.912 | 0.947 | |

TABLE 4
Comprehensive classification performance
when an RNN-Attention model is used

| | | N | V | Q | S | F | Acc |
|---|---|---|---|---|---|---|---|
| Original | precision | 0.997 | 0.943 | 0.988 | 0.514 | 0.491 | |
| | recall | 0.980 | 0.945 | 0.991 | 0.935 | 0.760 | 0.977 |
| | F1-score | 0.988 | 0.944 | 0.990 | 0.664 | 0.596 | |
| Augmented | precision | 0.997 | 0.952 | 0.902 | 0.721 | 0.904 | |
| | recall | 0.973 | 0.944 | 0.999 | 0.987 | 0.969 | 0.973 |
| | F1-score | 0.985 | 0.948 | 0.948 | 0.833 | 0.935 | |

Finally, it should be noted that the above descriptions are only preferred embodiments of the present disclosure, and are not intended to limit the present disclosure. Although the present disclosure has been described in detail with reference to the foregoing embodiments, those skilled in the art may still modify the technical solutions described in the foregoing embodiments, or equivalently substitute some technical features thereof. Any modification, equivalent substitution, improvement, etc. within the spirit and principles of the present disclosure shall fall within the scope of protection of the present disclosure.

What is claimed is:

1. An electrocardiograph (ECG) signal enhancement method based on a novel generative adversarial network (GAN), comprising the following steps:
a) resampling a collected ECG signal to obtain resampled ECG signal data;
b) normalizing the resampled ECG signal data;
c) extracting a location of an R-wave of a normalized ECG signal by using a Pan_Tompkin detection algorithm, dividing the normalized ECG signal into heartbeats based on m sampling points before the R-wave and n sampling points after the R-wave to obtain a heartbeat dataset U={U₁, U₂, . . . , Uₜ, . . . , U_{Z}}, wherein Uₜ represents a t^{th} heartbeat record, tϵ{1, 2, . . . , Z}, Z represents a quantity of the heartbeats, a length of each heartbeat record is represented by N, and each heartbeat record has 1*N dimensions;
d) dividing the heartbeat dataset U into a training set Xₜᵣₐᵢₙ, a validation set Xᵥₐₗ, and a test set Xₜₑₛₜ in a ratio of a:b:c;
e) establishing a GAN model comprising a generator network and a discriminator network, and inputting random noise Noise_{fixed} into the generator network of the GAN model in batches to output a synthesized signal set X_{g}, wherein the random noise Noise_{fixed} has batch*1*N dimensions;
f) inputting the training set Xₜᵣₐᵢₙ into the discriminator network of the GAN model to obtain an output label Xₜᵣₐᵢₙ^{d}, and inputting the synthesized signal set X_{g} into the discriminator network of the GAN model to obtain an output label X_{g}^{d};
g) training the generator network and the discriminator network by means of a game; and
h) inputting the random noise N_{g} with the batch*1*N dimensions into a trained generator network to obtain an enhanced ECG signal U_{g}.

2. The ECG signal enhancement method based on the novel GAN according to claim 1, wherein a sampling frequency during the resampling in the step a) is 125 Hz.

3. The ECG signal enhancement method based on the novel GAN according to claim 1, wherein in the step d), a=7, b=1.5, and c=1.5.

4. The ECG signal enhancement method based on the novel GAN according to claim 1, wherein the step e) comprises the following substeps:
e-1) constituting the generator network by a first convolutional block, a second convolutional block, a third convolutional block, a multi-branch bi-directional long short-term memory (BiLSTM) neural network module, an EEA-Net module, and a fully connected layer in sequence;
e-2) constituting the first convolutional block by a convolutional layer, a BatchNorm1d layer, and a Swish activation function layer in sequence, inputting the random noise Noise_{fixed} into the convolutional layer of the first convolutional block to obtain a feature X₁₋₁, inputting the feature X₁₋₁ into the BatchNorm1d layer of the first convolutional block to obtain a feature X₁₋₂, and inputting the feature X₁₋₂ into the Swish activation function layer of the first convolutional block to obtain a feature X₁;
e-3) constituting the second convolutional block by a convolutional layer, a BatchNorm1d layer, and a Swish activation function layer in sequence, inputting the feature X₁ into the convolutional layer of the second convolutional block to obtain a feature X₂₋₁, inputting the feature X₂₋₁ into the BatchNorm1d layer of the second convolutional block to obtain a feature X₂₋₂, and inputting the feature X₂₋₂ into the Swish activation function layer of the second convolutional block to obtain a feature X₂;
e-4) constituting the third convolutional block by a convolutional layer, a BatchNorm1d layer, a Swish activation function layer, and a Max-pooling layer in sequence, inputting the feature X₂ into the convolutional layer of the third convolutional block to obtain a feature X₃₋₁, inputting the feature X₃₋₁ into the BatchNorm1d layer of the third convolutional block to obtain a feature X₃₋₂, stitching the feature X₃₋₂ and the feature X₁₋₂, inputting a stitched feature into the Swish activation function layer of the third convolutional block to obtain a feature X₃₋₃, and inputting the feature X₃₋₃ into the Max-pooling layer of the third convolutional block to obtain a feature X₃;
e-5) constituting the multi-branch BiLSTM neural network module of the generator network by a first BiLSTM network, a second BiLSTM network, and a third BiLSTM network; inputting the feature X₃ into the first BiLSTM network, and calculating an output X^{bilstm1} of the first BiLSTM network according to a formula X^{bilstm1}=concat(h_{L,1},h_{R,1}), wherein concat(·) represents a stitching operation, h_{L,1} represents a hidden state of a last time step of forward propagation of the first BiLSTM network, and h_{R,1} represents a hidden state of a last time step of backward propagation of the first BiLSTM network; inputting the feature X₃ into the second BiLSTM network, and calculating an output X^{bilstm2} of the second BiLSTM network according to a formula X^{bilstm2}=concat(concat(h_{L,1},h_{L,2}),h_{R,2}, wherein h_{L,2} represents a hidden state of a last time step of forward propagation of the second BiLSTM network, and h_{R,2} represents a hidden state of a last time step of backward propagation of the second BiLSTM network; inputting the feature X₃ into the third BiLSTM network, and calculating an output X^{bilstm3} of the third BiLSTM network according to a formula X^{bilstm2}=concat(concat(h_{L,2},h_{L,3}),h_{R,3}), wherein h_{L,3} represents a hidden state of a last time step of forward propagation of the third BiLSTM network, and h_{R,3} represents a hidden state of a last time step of backward propagation of the third BiLSTM network; and calculating a final output feature X₄ of the multi-branch BiLSTM neural network module according to a formula X₄=concat(X^{bilstm1},X^{bilstm2},X^{bilstm3});
e-6) constituting the EEA-Net module of the generator network by a first convolutional block, a second convolutional block, an adaptive average pooling layer, a third convolutional block, and a fully connected module, wherein the first convolutional block is constituted by a convolutional layer and a LeakyReLU activation function layer in sequence, the second convolutional block is constituted by an adaptive convolutional layer and a LeakyReLU activation function layer in sequence, the third convolutional block is constituted by a convolutional layer and a LeakyReLU activation function layer in sequence, and the fully connected module is constituted by a fully connected layer and a Sigmoid activation function layer in sequence;
e-7) inputting the feature X₄ into the first convolutional block of the EEA-Net module of the generator network to obtain a feature X₄₋₁, inputting the feature X₄ into the second convolutional block of the EEA-Net module of the generator network to obtain a feature X₄₋₂, stitching the feature X₄₋₁ and the feature X₄₋₂ to obtain a stitching result C₁, inputting the stitching result C₁ into the adaptive average pooling layer, the third convolutional block, and the fully connected module of the EEA-Net module in sequence to obtain feature information X_{E}, and performing point multiplication on the feature information X_{E} and the feature X₄ to obtain a final output feature X₅ of the EEA-Net module; and
e-8) inputting the output feature X₅ into the fully connected layer of the generator network to obtain the synthesized signal set X_{g}.

5. The ECG signal enhancement method based on the novel GAN according to claim 4, wherein the convolutional layer of the first convolutional block in the step e-2) has a 1×3 convolution kernel and a stride of 1, and a padding of 1; the convolutional layer of the second convolutional block in the step e-3) has a 1×5 convolution kernel and a stride of 2, and a padding of 2; the convolutional layer of the third convolutional block in the step e-4) has a 1×3 convolution kernel and a stride of 1, and a padding of 1; in the step e-5), the first BiLSTM network has 64 neurons, the second BiLSTM network has 128 neurons, and the third BiLSTM network has 256 neurons; the convolutional layer of the first convolutional block in the step e-6) has a 1×3 convolution kernel and a stride of 1, and a padding of 1; the adaptive convolutional layer of the second convolutional block in the step e-6) has a stride of 1, and a padding of 1; and the convolutional layer of the third convolutional block in the step e-6) has a 1×5 convolution kernel and a stride of 1, and a padding of 1.

6. The ECG signal enhancement method based on the novel GAN according to claim 1, wherein the step f) comprises the following substeps:
f-1) constituting the discriminator network of the GAN model by a BiLSTM network, a first fully connected layer, a first Swish activation function layer, a second fully connected layer, a second Swish activation function layer, a Dropout layer, a third fully connected layer, and a Sigmoid layer in sequence;
f-2) inputting the training set Xₜᵣₐᵢₙ into the BiLSTM network of the discriminator network in batches to obtain a feature X₆, and inputting the feature X₆ into the first fully connected layer, the first Swish activation function layer, the second fully connected layer, the second Swish activation function layer, the Dropout layer, the third fully connected layer, and the Sigmoid layer of the discriminator network in sequence to obtain the output label Xₜᵣₐᵢₙ^{d}; and
f-3) inputting the synthesized signal set X_{g} into the BiLSTM network of the discriminator network in batches to obtain a feature X₇, and inputting the feature X₇ into the first fully connected layer, the first Swish activation function layer, the second fully connected layer, the second Swish activation function layer, the Dropout layer, the third fully connected layer, and the Sigmoid layer of the discriminator network in sequence to obtain the output label X_{g}^{d}.

7. The ECG signal enhancement method based on the novel GAN according to claim 6, wherein in the step f-1), the BiLSTM network has 128 hidden neurons, and the Dropout layer has a random dropout rate of 0.2.

8. The ECG signal enhancement method based on the novel GAN according to claim 6, wherein the step g) comprises the following substeps:
g-1) constructing a real label Lᵣₑₐₗ containing only 1s, and a false label L_{fake} containing only 0s;
g-2) calculating a loss function loss_{D} of the discriminator network according to a following formula:
$${loss}_{D} = - ( {L}_{real} * log ( {X}_{train}^{d} ) + ( 1 - {L}_{real} ) * log ( 1 - {X}_{train}^{d} ) ) - ( {L}_{fake} * log ( {X}_{g}^{d} ) + ( 1 - {L}_{fake} ) * log ( 1 - {X}_{g}^{d} ) ) ;$$
g-3) calculating a loss function loss_{G} of the generator network according to a following formula:
$${loss}_{G} = β [ - ( {L}_{real} * log ( {X}_{g}^{d} ) + ( 1 - {L}_{real} ) * log ( 1 - {X}_{g}^{d} ) ) ] + γ [ \frac{1}{K} {\sum }_{i = 1}^{K} {( {X}_{train}^{i} - {X}_{g}^{i} )}^{2} ] ,$$
wherein both β and γ are equilibrium factors, Xₜᵣₐᵢₙⁱ represents an i^{th} heartbeat record in the training set Xₜᵣₐᵢₙ, X_{g}ⁱ represents an i^{th} record in the synthesized signal set X_{g}, iϵ{1, 2, . . . , K}, and
$$K = \frac{Za}{a + b + c} ;$$
and
g-4) using an Adam optimizer to train the generator network based on the loss function loss_{G} of the generator network, and train the discriminator network based on the loss function loss_{D} of the discriminator network, to obtain an optimized GAN model.

9. The ECG signal enhancement method based on the novel GAN according to claim 8, wherein β=0.7, and γ=0.2.

10. The ECG signal enhancement method based on the novel GAN according to claim 8, wherein during the training in the step g-4), a learning rate, a batch size, and an iteration quantity are respectively set to 0.0002, 128, and 2500 for both the generator network and the discriminator network, and the learning rates of the generator network and the discriminator network are updated to 0.1 times the original ones every 500 iterations.
